(19) United States
(12) Patent Application Publication
(10) Pub. No.: US 2017/0046480 A1
(43) Pub. Date: Feb. 16, 2017

(54) DEVICE AND METHOD FOR DETECTING THE PRESENCE OR ABSENCE OF NUCLEIC ACID AMPLIFICATION

(71) Applicant: Tetracore, Inc., Rockville, MD (US)

(72) Inventors: David R. Almassian, Gaithersburg, MD (US); Jonathan Yu, Gaithersburg, MD (US)

(73) Assignee: Tetracore, Inc.

(21) Appl. No.: 15/235,573

(22) Filed: Aug. 12, 2016

Related U.S. Application Data
(60) Provisional application No. 62/205,251, filed on Aug. 14, 2015.

(51) Int. Cl. G06F 19/24; C12Q 1/68

(52) U.S. Cl. CPC G06F 19/24; C12Q 1/682

(57) ABSTRACT

Methods and apparatus are disclosed detecting the presence or absence of nucleic acid amplification employing classification of the features of a curve representing the DNA amplification reporter signal, and calculating the probability of nucleic acid amplification being present at a predetermined thermal cycle.

### PRIORITY CLAIM

This application claims priority from U.S. Provisional Patent Application No. 62/205,251 filed on Aug. 14, 2015, which is hereby incorporated by reference in its entirety in the present application.

### TECHNICAL FIELD

The present disclosure relates generally to a method of detecting the presence or absence of nucleic acid amplification.

### BACKGROUND

During various scientific and medical procedures, there is often a need to detect the presence or absence of one or more target DNA sequences (“target sequences”) in a pool of many DNA sequences.

This is typically done by first amplifying the nucleic acid, such as through Polymerase Chain Reactions (“PCRs”) or through isothermal reactions (e.g., RPA, HDA, LAMP, NASBA, RCA, ICAN, SMART, SDA). This process involves detecting the products of nucleic acid amplification during the reaction (i.e., in real-time).

PCRs are reactions wherein a DNA assay is run through multiple thermal cycles. In each cycle, when a sufficient temperature is reached, hydrogen bonds between complementary bases are disrupted due to DNA melting, yielding single-stranded DNA molecules. When the temperature in a given cycle is lowered, primers anneal to the single-stranded DNA molecules if the primer sequence closely matches the sequence complementary to the single-stranded DNA molecules. When the temperature is increased again, the primer synthesizes a new DNA strand complementary to the single-stranded DNA molecule. This leads to an exponential increase of target sequences and may be detected using, for example, various probes (e.g., fluorescent DNA probes).

In the case of PCR, the presence of nucleic acid amplification is typically accomplished by exciting a probe reporter with a laser or LED and monitoring the probe for fluorescence while cycling an assay though thermal cycles. The intensity of the fluorescence is then analyzed to determine the presence or absence of the nucleic acid amplification. In many cases, nucleic acid amplification further indicates the presence or absence of the target sequence. Electrochemical and electrical detection processes are also known. See e.g., Goda et al., “Electrical and Electrochemical Monitoring of Nucleic Acid Amplification,” Front, Bioeng. Biotecnol, 2015; 3: 29 (2015).

A linear threshold is typically set such that the presence of nucleic acid amplification is inferred when the intensity of the fluorescence increases above the threshold (for an increasing fluorescence detection signal) or decreases below the threshold (for a decreasing fluorescence detection signal). The linear threshold is typically set by the operator based on experience with a particular assay, or may be specified by the assay manufacturer. In an illustrative embodiment the linear threshold is set slightly above the system noise floor.

Use of a linear threshold to infer the presence of nucleic acid amplification has various drawbacks, including detecting false positives and false negatives. False positive detections may be caused, for instance, by an upward drift in in the fluorescence detection signal over time or a rapid linear drift in the fluorescence detection signal at the beginning of a reaction, even in the absence of nucleic acid amplification. An attempt to compensate for the drift by adjusting the linear threshold may result in false negatives.

Additionally, false positive and false negative detections may result from the fact that different biological assays produce fluorescence detection signals of varying strengths. A threshold that is appropriate for one assay may lead to false positive or false negative detections in another assay.

Another drawback of using a linear threshold to infer the presence of nucleic acid amplification is the necessity of adjusting the threshold to account for variances in the sensitivity of the instruments used to detect the fluorescence.

All the foregoing adjustments of the linear threshold require time and effort. Failure to expend the time and effort could result in false positive and negative detections when using a linear threshold.

The disclosed methods and apparatus are directed to overcoming one or more of the problems set forth above and/or other problems or shortcomings in the prior art.

### SUMMARY

The present disclosure is directed to a method for detecting the presence or absence of nucleic acid amplification.

Consistent with at least one disclosed embodiment, a method is disclosed for detecting nucleic acid amplification. In one embodiment, this may be accomplished by initiating a PCR and including a probe in the reaction mixture.

Amplification detection may also include detecting an original reporter signal, which corresponds to the intensity of the reporter fluorescence.

Amplification detection may also include smoothing an original reporter signal,

Amplification detection may also include creating residual noise data by subtracting the smoothed reporter signal from the original reporter signal.

Amplification detection may also include creating many randomized residual noise datasets by sampling, with replacement, the residual noise data, whereby each randomized residual noise dataset has the same size as the residual noise data.

Amplification detection may also include creating many input datasets by adding the randomized residual noise datasets to the smoothed reporter signal.

Amplification detection may also include using a trained machine learning system to classify each input dataset as indicating the presence or absence of nucleic acid amplification.

Amplification detection may also include, in at least the case of a PCR, determining, for each input dataset classified as indicating the presence of nucleic acid amplification, at which thermal cycle in each input dataset nucleic acid amplification was present.

Amplification detection may also include, in at least the case of a PCR, determining the thermal cycle at which nucleic acid amplification is believed to be present.

Amplification detection may also include inferring, from the classifications of all input datasets, the probability that nucleic acid amplification was present. This may be done, for example, by dividing the number of input datasets with a thermal cycle at which nucleic acid amplification was determined to be present near the thermal cycle at which nucleic acid amplification is believed to be present by the total number of input datasets. In an illustrative embodiment, +/−1 CT from the CT under consideration are included for PCR.

According to an aspect of the present disclosure, assay product development as described herein advantageously allows the assay to be developed without especial concern about threshold adjustments. In manufacturing of both the instrument and the assay, one aspect of the present disclosure allows for more tolerance and/or less precision in the fluorescence range without affecting false-positive or false-negative rates.

According to another aspect of the present disclosure, assays conducted as described herein advantageously exhibit reduced variance, allowing more consistent/repeatable assay results.

Other embodiments of this disclosure are disclosed in the accompanying drawings, description, and claims. Thus, this summary is exemplary only, and is not to be considered restrictive.

### BRIEF DESCRIPTION OF DRAWINGS

The accompanying drawings, which are incorporated in and constitute a part of this specification, illustrate the disclosed embodiments and together with the description, serve to explain the principles of the various aspects of the disclosed embodiments. In the drawings:

FIG. 1: Illustrates an exemplary original reporter signal.

FIG. 2: Illustrates an exemplary smoothed reporter signal.

FIG. 3: Illustrates exemplary residual noise data.

FIG. 4: Illustrates an exemplary randomized residual noise dataset.

FIG. 5: Illustrates an exemplary input dataset.

FIG. 6: Illustrates an exemplary process for detecting the presence or absence of nucleic acid amplification.

It is to be understood that both the foregoing general description and the following detailed description are exemplary and explanatory only and are not restrictive of the claims.

### DESCRIPTION OF EXEMPLARY EMBODIMENTS

Reference will now be made to certain embodiments consistent with the present disclosure, examples of which are illustrated in the accompanying drawings. Wherever possible, the same reference numbers are used throughout the drawings to refer to the same or like parts.

The present disclosure describes a method of detecting nucleic acid amplification in a pool of DNA sequences.

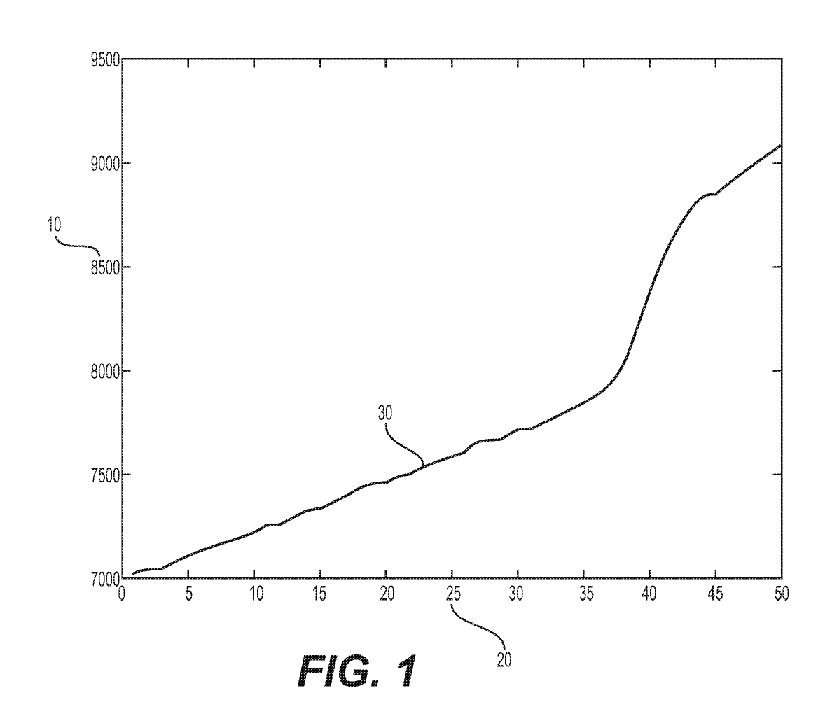

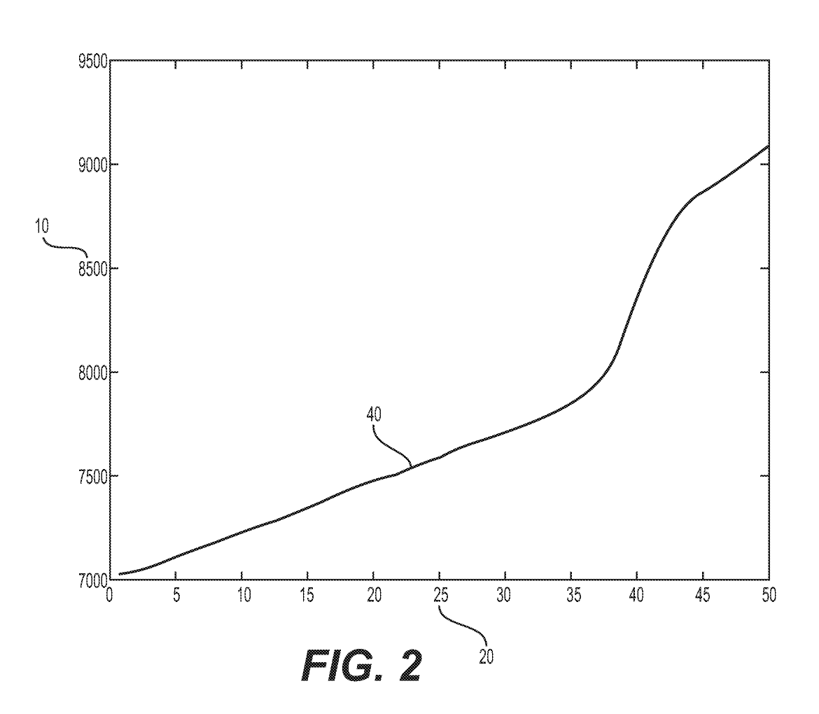

Detecting nucleic acid amplification may be accomplished by attempting to initiate a nucleic acid amplification reaction, such as a PCR, and, for example, detecting, using a probe in the PCR mixture, an original reporter signal, which corresponds to the intensity of the reporter fluorescence. FIG. 1 shows an exemplary embodiment of an original reporter signal 30, graphed against horizontal axis 20 and vertical axis 10, representing the thermal cycle at which the reporter signal was collected and the strength of the reporter signal, respectively. In exemplary embodiments, the original reporter signal 30 may, for example, be smoothed to create a smoothed reporter signal. FIG. 2 shows an exemplary embodiment of a smoothed reporter signal 40 graphed against horizontal axis 20 and vertical axis 10,

In exemplary embodiments, the original reporter signal 30 would vary depending on, among other things, the type of probe used. For example, by exciting a fluorescent DNA probe reporter with a laser or LED and monitoring the probe for fluorescence while cycling an assay though thermal cycles, one may receive an indication of whether nucleic acid amplification is present. The original reporter signal 30 may be acquired by, for example, measuring one or more attributes of the probe reporter, including, for example, when the probe reporter is excited with a laser or LED.

Smoothing of the original reporter signal 30 may be accomplished by, for example, running the signal through a low pass filter or any other system capable of signal smoothing, including but not limited to any of, or any combination of, a digital, analog, mixed, and software system. FIG. 2 shows an exemplary smoothed reporter signal 40. Exemplary smoothing and curve-fitting methods usable with the present disclosure include those described in O'Haver et al., “A Pragmatic Introduction to Signal Processing” University of Maryland, 2015. PDF e-book. The contents of this document are incorporated herein by reference in its entirety.

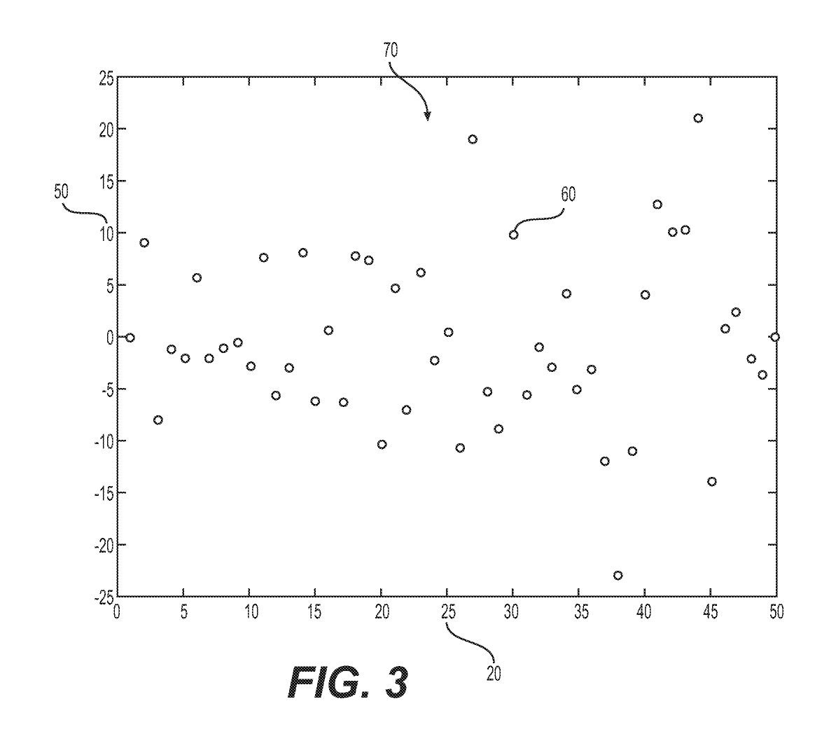

Amplification detection may also include creating residual noise data 70, such as that shown in FIG. 3, by subtracting the smoothed reporter signal 40 from the original reporter signal 30. This may be done using a system including but not limited to any of, or any combination of, a digital, analog, mixed, and software system. The residual noise data 70 in FIG. 3 is graphed against horizontal axis 20 and vertical axis 50, the latter representing the difference in reporter signal strength between the original reporter signal 30 and the smoothed reporter signal 40 at each thermal cycle indicated on horizontal axis 20.

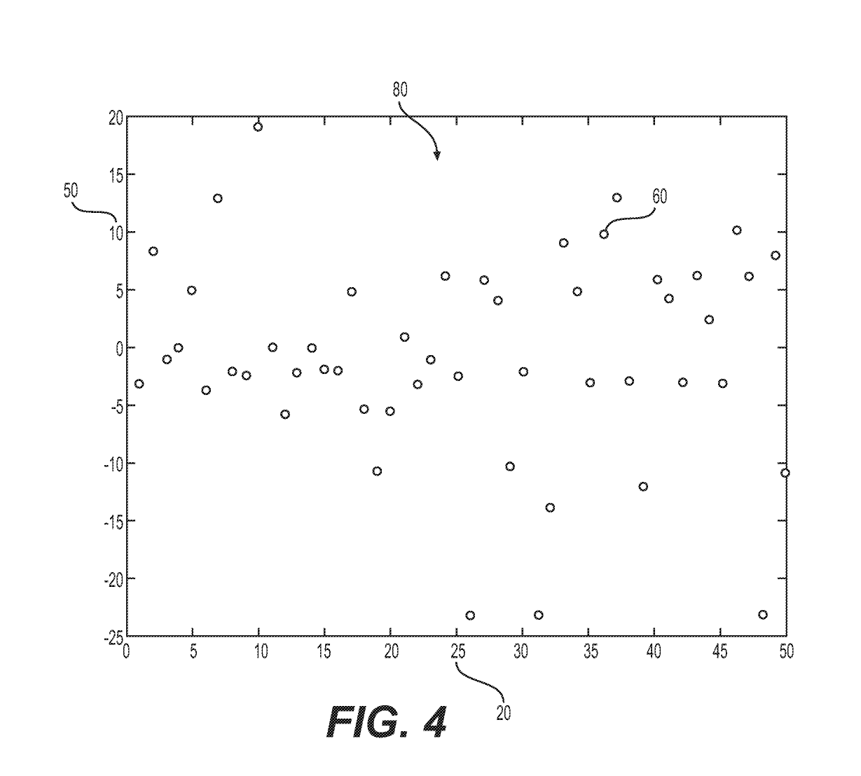

Amplification detection may also include creating many randomized residual noise datasets, such as the randomized residual noise dataset 80, shown in FIG. 4, by sampling, with replacement, the residual noise data 70, such as that shown in FIG. 3 and FIG. 4, whereby each randomized residual noise dataset 80 has the same size as the residual noise data 70. In at least one embodiment, randomized residual noise dataset 80 may be comprised of residuals such as residual 60, wherein each residual for a given cycle is a randomly selected, with replacement, residual from the residual noise data 70.

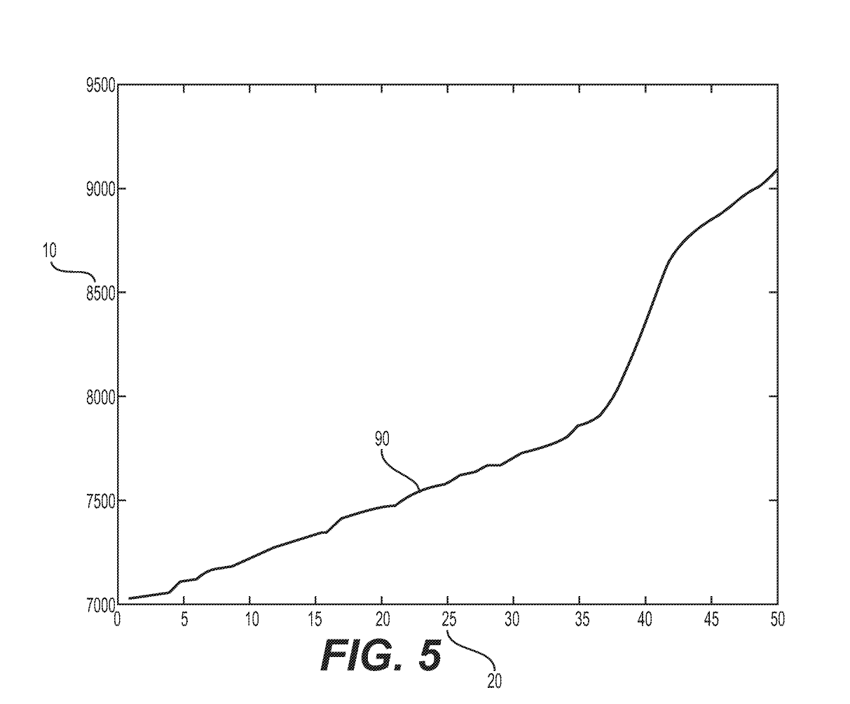

Amplification detection may also include creating many input datasets, such as the input dataset 90 shown in FIG. 5, by adding many randomized residual noise datasets, such as randomized residual noise dataset 80, to the smoothed reporter signal 40. This may be done using a system including but not limited to any of, or any combination of, a digital, analog, mixed, and software system.

Amplification detection may also include extracting quantitative features from each input dataset 90. In one embodiment, the quantitative feature extracted from the input datasets, such as input dataset 90, may include a measure of curvature of the input dataset 90. The measure of curvature may be calculated, for example, by connecting the first and last points of the curve with a straight line and measuring the difference in signal strength between each point of the straight line and the corresponding point of the curve. The largest difference in signal strength between each point of the straight line and the corresponding point of the curve is used as the measure of curvature, and the location of the largest difference is used as the potential CT value. In another exemplary embodiment, the application can employ the peak of the second derivative wherein the second derivative of the smoothed curve is calculated and then subject to a peak-detection evaluation.

In one embodiment, the quantitative feature extracted from the input datasets, such as input dataset 90, may include the quotient of the difference between the signal strength at the last point in the input dataset 90 and the signal strength at the potential CT value in the input dataset 90 divided by the average signal strength of the first five points in the input dataset.

In one embodiment, the quantitative feature extracted from the input datasets, such as input dataset 90, may include the signal strength of the peak of the second derivative of the curve representing the input dataset.

In exemplary embodiments, quantitative feature extraction from the input datasets, such as input dataset 90, or the training data may be done by a processor configured to execute instructions contained in memory to implement a DSP method that extracts quantitative features from the datasets.

Amplification detection may also include using a trained machine learning system to classify each input dataset 90 as indicating the presence or absence of nucleic acid amplification.

The machine learning system may be a support vector machine. The machine learning system may be trained using training data based on previous nucleic acid amplification detections that yielded results with a high degree of certainty.

In exemplary embodiments, the machine learning system may include a classifier that provides a mathematical function for mapping (or classifying) a vector of quantitative features extracted from the input datasets, such as input dataset 90, into one or more predefined classifications. The classifications may represent whether nucleic acid amplification is present or not present. The classifiers may be built by forming at least one training dataset, wherein each piece of data is assigned a classification.

In exemplary embodiments, the process of building a classifier from training data may involve the selection of a subset of quantitative features (from the set of all quantitative features), along with the construction of a mathematical function which uses these features as input and which produces as its output an assignment of the input dataset 90 to a specific class. The mathematical function may have coefficients that relate to one another in a manner specified at least in part by at least one training dataset. After a classifier is built, it may be used to classify unlabeled datasets as belonging to one or the other class. Classification accuracy is then reported using testing data which may or may not overlap with the training data, but for which a priori classification data is also available. The accuracy of the classifier is dependent upon the selection (or “picking”) of quantitative features that comprise part of the specification of the classifier (i.e., selection of quantitative features that contribute most to the classification task ensures the best classification performance).

In exemplary embodiments, the machine learning system's training data may be sampled many times to create multiple distinct training datasets. At least one of the input datasets, such as input dataset 90, may be run through the machine learning system and classified using a classifier trained with at least one of the training datasets,

In exemplary embodiments, the trained machine learning system classifies quantitative features extracted from each input dataset, such as input dataset 90 shown in FIG. 5. The machine learning system may be trained with training data comprising at least one quantitative feature extracted from input datasets derived from original reporter signals, such as original reporter signal 30 in FIG. 1, in previous nucleic acid amplification detections that yielded results with a high degree of certainty.

Amplification detection may also include, in the case of at least a PCR, for example, determining, for each input dataset, such as input dataset 90, classified as indicating the presence of nucleic acid amplification, at which thermal cycle in each input dataset nucleic acid amplification was present.

In exemplary embodiments, analysis of input dataset 90 may be done by a processor configured to execute instructions contained in memory to implement a DSP method that classifies input datasets as indicating the presence or absence of nucleic acid amplification.

Amplification detection may also include, in at least the case of a PCR, determining the thermal cycle at which nucleic acid amplification was believed to be present.

Amplification detection may also include inferring, from the classifications of all input datasets, such as input dataset 90, the probability that nucleic acid amplification was present. This may be done, for example, by dividing the number of input datasets with a thermal cycle at which nucleic acid amplification was determined to be present near the thermal cycle at which nucleic acid amplification is believed to be present by the total number of input datasets.

In exemplary embodiments, the nucleic acid amplification may occur in an isothermal reaction. Exemplary embodiments can employ Recombinase Polymerase Amplification (RPA), Helicase-Dependent Amplification (HDA), Loop-mediated isothermal amplification (LAMP), Nucleic Acid Sequence Based Amplification (NASBA), Rolling Circle Amplification (RCA), Isothermal and Chimeric primer-initiated Amplification of Nucleic acids (ICAN), SMART™, Strand Displacement Amplification (SDA), among others, including electrochemical and electrical processes.

An aspect of the present disclosure is a method of budding a classifier for classification of individual input data into one of two or more categories, each indicating the presence or absence of nucleic acid amplification. The method comprises the steps of providing a processor configured to build a classifier, and providing a memory device operatively coupled to the processor, wherein the memory device stores one or more datasets comprising a collection of quantitative features extracted from the results of nucleic acid amplification detections wherein the results were obtained with a high degree of certainty. The processor is configured to select a plurality of features from input datasets, such as input dataset 90, and one or more other features from the datasets comprising a collection of quantitative features extracted from the input datasets of nucleic acid amplification detections wherein the results, such as the presence or absence of nucleic acid amplification, were obtained with a high degree of certainty, constructing a classifier using the latter selected quantitative features, and evaluating performance of the classifier using input datasets, such as input dataset 90, assigned a priori to one of the two categories.

In a further illustrative embodiment, the input can be bootstrapped while using a linear threshold. Using this approach, the input could be resampled but the assay could proceed using a linear threshold rather than searching for the features of the resampled input. While such an approach might not benefit all processes, it could be beneficial in certain instances, such as if there is a large amount of pre-processing (smoothing, baseline, etc.) performed before the linear threshold is applied.

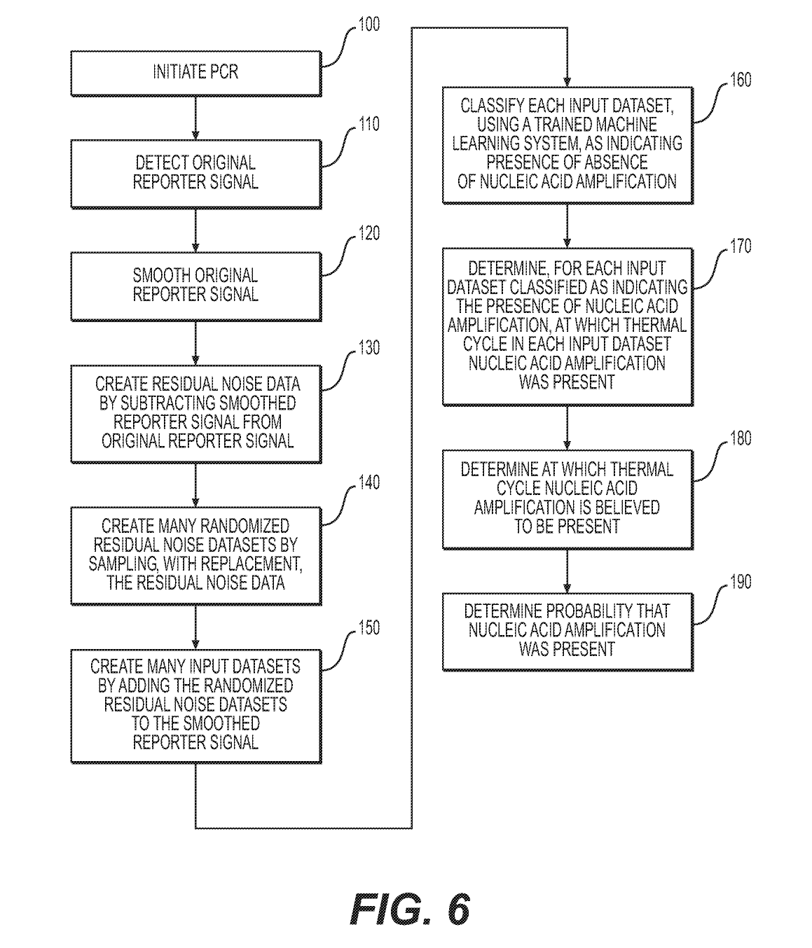

In an exemplary embodiment, the presence or absence of nucleic acid amplification may be determined using the process illustrated in FIG. 6. At step 100, one or more method users would initiate a PCR. At step 110, the one or more users would detect an original reporter signal, such as original reporter signal 30. At step 120, the one or more users would smooth the original reporter signal, resulting in a smoothed reporter signal, such as smoothed reporter signal 40. At step 130, the one or more users would subtract the smoothed reporter signal from the original reporter signal, resulting in residual noise data, such as residual noise data 70. At step 140, the one or more users would create many randomized residual noise datasets, such as randomized residual noise dataset 80, by sampling, with replacement, the residual noise data. At step 150, the one or more users would create many input datasets, such as input dataset 90, by adding the randomized residual noise datasets to the smoothed reporter signal. At step 160, the one or more users would classify each input dataset, using a trained machine learning system, as indicating the presence or absence of nucleic acid amplification. At step 170, the one or more users would determine, for each input dataset classified as indicating the presence of nucleic acid amplification, at which thermal cycle in each input dataset nucleic acid amplification was present. At step 180, the one or more users would determine at which thermal cycle nucleic acid amplification is believed to be present. At step 190, the one or more users would determine the probability that nucleic acid amplification was present by dividing the number of input datasets with a thermal cycle at which nucleic acid amplification was determined to be present near the thermal cycle at which nucleic acid amplification is believed to be present by the total number of input datasets.

The foregoing description has been presented for purposes of illustration. It is not exhaustive and is not limited to the precise forms or embodiments disclosed. Modifications and adaptations will be apparent to those skilled in the art from consideration of the specification and practice of the disclosed embodiments.

Moreover, while illustrative embodiments have been described herein, the scope of any and all embodiments include equivalent elements, modifications, omissions, combinations (e.g., of aspects across various embodiments), adaptations and/or alterations as would be appreciated by those skilled in the art based on the present disclosure. The limitations in the claims are to be interpreted broadly based on the language employed in the claims and not limited to examples described in the present specification or during the prosecution of the application. The examples are to be construed as non-exclusive. Furthermore, the steps of the disclosed methods may be modified in any manner, including by reordering steps and/or inserting or deleting steps. It is intended, therefore, that the specification and examples be considered as illustrative only, with a true scope and spirit being indicated by the following claims and their full scope of equivalents.

What is claimed is:

1. A method of detecting the presence or absence of nucleic acid amplification, comprising:
bootstrapping/resampling input data to a machine learning method, wherein the machine learning method calculates classifications;
classifying the features of a curve representing the DNA amplification reporter signal,
determining the probability of the presence or absence of nucleic acid amplification from the classifications, and
determining the probability of nucleic acid amplification being present at a predetermined thermal cycle.

2. The method of detecting the presence or absence of nucleic acid amplification of claim 1, wherein the reporter signal is acquired by measuring one or more attributes of the probe reporter.

3. The method of detecting the presence or absence of nucleic acid amplification of claim 1, wherein the reporter signal is smoothed.

4. The method of detecting the presence or absence of nucleic acid amplification of claim 1, wherein the amplification further includes creating residual noise data.

5. The method of detecting the presence or absence of nucleic acid amplification of claim 1, wherein the amplification detection includes creating at least one randomized residual noise dataset.

6. The method of detecting the presence or absence of nucleic acid amplification of claim 1, wherein the amplification detection includes extracting quantitative features from an input dataset.

7. The method of detecting the presence or absence of nucleic acid amplification of claim 6, wherein the quantitative feature extracted from an input dataset includes the signal strength of the peak of the second derivative of a curve representing the input dataset.

8. A machine learning method including bootstrapping or resampling input data to the machine learning method, wherein the machine learning method calculates classifications, the method comprising the steps of:
smoothing/curve fitting the input data;
calculating the residuals to the smoothed/curve fit input data;
randomly sampling from the residuals;
creating many input datasets by adding the randomly sampled residuals to the smoothed/curve fit input data; and
applying the machine learning method to the many input datasets.

8. The machine learning method of claim 8, further comprising building a classifier from training data.

9. The machine learning method of claim 9, further comprising selecting a subset of quantitative features from the set of all quantitative features.

10. The machine learning method of claim 9, wherein the selected subset of quantitative features derived from reporter signals in previous amplification detections that yielded results with a high degree of certainty.

11. The machine learning method of claim 8 wherein the input is bootstrapped using a linear threshold.
